SEQUENCE LISTING (6 sequences)

SEQ ID NO: 1 - DNA, 1191 bp, Haemophilus influenzae
atgcaaaaacaaaacattgtcattcttggttcaacgggatcaatcggtaagagtaccctttctgttatcgaaaataaccctcagaaatatcatgcatttgcactcgtaggcggaaaaaatgtagaagcaatgtttgaacaatgtatcaaattccgaccgcactttgcggctcttgatgatgtaaatgcggctaaaattttacgtgaaaaattaattgcgcatcatattccaacggaagtattagcaggacgacgagctatttgcgaactcgcagcacacccagatgccgatcagataatggcgtcgattgttggtgcagcaggattgttaccgactctttcagcggttaaagcaggtaaacgggtattactggcaaataaagaatcactggtaacctgcggacagctttttattgatgccgtaaaaaactatggctcgaagcttttaccagtagatagtgaacataatgctatctttcaatcattaccgccagaagcacaagaaaaaatcggtttttgcccactttctgaattaggtgtaagtaaaattatactcactggttctggcggaccattccgttacacgccacttgaacaattcaccaacataacaccagaacaagcggttgcacaccctaattggtctatgggtaaaaaaatttctgtcgattcagctacaatgatgaataagggcttggaatacattgaggctcgctggcttttcaatgcaagtgcggaagaaatggaagttattattcatccacaatcaattattcattctatggtacggtatgttgacggctcagtcattactcaaatgggaaatccagatatgcgtacaccaattgcagaaactatggcatatcctcaccgcacttttgctggagtagaaccactcgatttctttaaaatcaaagaactgacatttattgaacctgattttaatcgctatccaaatttaaaactggctattgatgcctttgctgcgggtcaatatgcgacaacagcaatgaatgcagctaatgaaattgccgtacaagcatttttagatcgtcaaattggctttatggatattgcaaaaattaattcgaaaacaattgagagaatttcgccttataccattcaaaatattgatgatgtactcgaaattgatgcacaagcaagagagattgcgaaaacactacttagagaa

SEQ ID NO: 2 - protein, 1588 aa, Haemophilus influenzae
MQKQNIVILGSTGSIGKSTLSVIENNPQKYHAFALVGGKNVEAMFEQCIKFRPHFAALDDVNAAKILREKLIAHHIPTEVLAGRRAICELAAHPDADQIMASIVGAAGLLPTLSAVKAGKRVLLANKESLVTCGQLFIDAVKNYGSKLLPVDSEHNAIFQSLPPEAQEKIGFCPLSELGVSKIILTGSGGPFRYTPLEQFTNITPEQAVAHPNWSMGKKISVDSATMMNKGLEYIEARWLATGCAAAAACAAAACATTGTCATTCTTGGTTCAACGGGATCAATCGGTAAGAGTACCCTTTCTGTTATCGAAAATAACCCTCAGAAATATCATGCATTTGCACTCGTAGGCGGAAAAAATGTAGAAGCAATGTTTGAACAATGTATCAAATTCCGACCGCACTTTGCGGCTCTTGATGATGTAAATGCGGCTAAAATTTTACGTGAAAAATTAATTGCGCATCATATTCCAACGGAAGTATTAGCAGGACGACGAGCTATTTGCGAACTCGCAGCACACCCAGATGCCGATCAGATAATGGCGTCGATTGTTGGTGCAGCAGGATTGTTACCGACTCTTTCAGCGGTTAAAGCAGGTAAACGGGTATTACTGGCAAATAAAGAATCACTGGTAACCTGCGGACAGCTTTTTATTGATGCCGTAAAAAACTATGGCTCGAAGCTTTTACCAGTAGATAGTGAACATAATGCTATCTTTCAATCATTACCGCCAGAAGCACAAGAAAAAATCGGTTTTTGCCCACTTTCTGAATTAGGTGTAAGTAAAATTATACTCACTGGTTCTGGCGGACCATTCCGTTACACGCCACTTGAACAATTCACCAACATAACACCAGAACAAGCGGTTGCACACCCTAATTGGTCTATGGGTAAAAAAATTTCTGTCGATTCAGCTACAATGATGAATAAGGGCTTGGAATACATTGAGGCTCGCTGGCTTTTCAATGCAAGTGCGGAAGAAATGGAAGTTATTATTCATCCACAATCAATTATTCATTCTATGGTACGGTATGTTGACGGCTCAGTCATTACTCAAATGGGAAATCCAGATATGCGTACACCAATTGCAGAAACTATGGCATATCCTCACCGCACTTTTGCTGGAGTAGAACCACTCGATTTCTTTAAAATCAAAGAACTGACATTTATTGAACCTGATTTTAATCGCTATCCAAATTTAAAACTGGCTATTGATGCCTTTGCTGCGGGTCAATATGCGACAACAGCAATGAATGCAGCTAATGAAATTGCCGTACAAGCATTTTTAGATCGTCAAATTGGCTTTATGGATATTGCAAAAATTAATTCGAAAACAATTGAGAGAATTTCGCCTTATACCATTCAAAATATTGATGATGTACTCGAAATTGATGCACAAGCAAGAGAGATTGCGAAAACACTACTTAGAGAAFNASAEEMEVIIHPQSIIHSMVRYVDGSVITQMGNPDMRTPIAETMAYPHRTFAGVEPLDFFKIKELTFIEPDFNRYPNLKLAIDAFAAGQYATTAMNAANEIAVQAFLDRQIGFMDIAKINSKTIERISPYTIQNIDDVLEIDAQAREIAKTLLRE

SEQ ID NO: 3 - DNA, 32 bp, Escherichia coli
ttatacatatgaagcaactcaccattctgggc

SEQ ID NO: 4 - DNA, 35 bp, Escherichia coli
ttataggatcctcagcttgcgagacgcatcacctc

SEQ ID NO: 5 - DNA, 34 bp, Haemophilus influenzae
tatatacatatgcaaaaacaaaacattgtcattc

SEQ ID NO: 6 - DNA, 40 bp, Haemophilus influenzae
tatataggatcctcattctctaagtagtgttttcgcaatc

(19) United States
(12) Patent Application Publication
(10) Pub. No.: US 2005/0096255 A1
(43) Pub. Date: May 5, 2005

(54) Methods of modulating activity of DXR

(75) Inventors: Deborah Dee Jaworski, Collegeville, PA (US); David J. Payne, Collegeville, PA (US); Courtney E. Slater-Radosti, Collegeville, PA (US); Kang Yan, Collegeville, PA (US)

Correspondence Address: GLAXOSMITHKLINE;Corporate Intellectual Property - UW2220, P.O. Box 1539, King of Prussia, PA, 19406-0939, US

(73) Assignee: SmithKline Beecham Corporation

(21) Appl. No.: 10/818,210

(22) Filed: Apr. 5, 2004

Related U.S. Application Data
(63) Continuation of application No. 09/925,778, filed on Aug. 9, 2001, now abandoned.
(60) Provisional application No. 60/223,909, filed on Aug. 9, 2000.

(51) Int. Cl. A61K 38/54; A61K 31/12; C12N 9/99; C12N 9/64

(52) U.S. Cl. 514/2; 435/184; 435/226; 514/690

(57) ABSTRACT

A method of modulating the activity of a Haemophilus influenzae DXR reductoisomerase enzyme, involving contacting a DXR reductoisomerase enzyme with a compound that inhibits non-mevalonate isoprenoid biosynthesis by lowering or preventing the biosynthesis of either menaquinone or ubiquinone. Pharmaceutical compositions containing such compounds may be used to treat infections. Also provided are methods for utilizing DXR polypeptides and polynucleotides to screen for antibacterial compounds.

### FIELD OF THE INVENTION

The invention relates generally to novel methods of treating infections, infestations and other mammalian diseases, particularly infections associated with pathogenic organisms, using compounds that inhibit a biological activity of polynucleotides and polypeptides of the Reductoisomerase family.

### BACKGROUND OF THE INVENTION

Infections caused by or related to Haemophilus influenzae are a major cause of human illness worldwide, and the frequency of resistance to standard antibiotics has risen dramatically over the last decade. Hence, there exists an unmet medical need and demand for new anti-microbial agents, vaccines, and drug screening methods.

Moreover, the drug discovery process is currently undergoing a fundamental revolution as it embraces “functional genomics,” that is, high throughput genome or gene-based biology. This approach is rapidly superseding earlier approaches based on “positional cloning” and other methods. Functional genomics relies heavily on the various tools of bioinformatics to identify gene sequences of potential interest from the many molecular biology databases now available as well as from other sources. There is a continuing and significant need to identify and characterize further genes and other polynucleotide sequences and their related polypeptides, as targets for drug discovery.

Clearly, there exists a need for polynucleotides and polypeptides, such as the DXR embodiments of the invention, that have a present benefit of, among other things, being useful to screen compounds for antimicrobial activity. Such factors are also useful to determine their role in pathogenesis of infection, dysfunction and disease. There is also a need for identification and characterization of such factors and their antagonists and agonists to find ways to prevent, ameliorate or correct such infection, dysfunction and disease.

### SUMMARY OF THE INVENTION

Proteins and polypeptides of the Reductoisomerase family, as well as their variants, are referred to herein as “DXR,” “DXR polynucleotide(s),” and “DXR polypeptide(s),” as the case may be.

The present invention relates to DXR, in particular H. influenzae DXR polypeptides and H. influenzae DXR polynucleotides, recombinant materials and methods for their production and use. In one aspect, the invention provides a method of modulating an activity of DXR reductoisomerase enzyme of H. influenzae comprising contacting said H. influenzae enzyme with a compound that inhibits non-mevalonate isoprenoid biosynthesis. In one embodiment of this method, compounds that inhibit the reductoisomerase enzyme thereby inhibiting non-mevalonate isoprenoid biosynthesis by lowering or preventing the biosynthesis of either menaquinone or ubiquinone or the biosynthesis of both menaquinone and ubiquinone. A further embodiment of this method is wherein the activity of DXR is selected from the group consisting of: formation of a dimer, use of manganese as a substrate by DXR, use of NADPH as substrate by DXR, NADPH binding to DXR prior to or simultaneous with manganese binding, isomerization of substrate, binding of substrate, reductoisomerizations, binding of DXR with a cellular component, conversion of 1-deoxy-D-xylulose-5-phosphate to 2C-methyl-D-erythritol-4-phosphate, conversion of NADPH to NADP, inhibition of DXR by fosmidomycin, fosfomycin, FR-33289, or FR-900098, and binding of fosmidomycin, fosfomycin, FR-33289, or FR-900098 to DXR. Another embodiment of the method is wherein said modulating is inhibiting. A preferred embodiment is provided wherein contacting said enzyme with said compound inhibits the biosynthesis of isoprenoids. In another embodiment of this method contacting said enzyme with said compound inhibits the biosynthesis of either menaquinone or ubiquinone or biosynthesis of both menaquinone and ubiquinone.

In still an additional preferred embodiment, it is provided wherein, the DXR reductoisomerase enzyme is selected from the group consisting of: (i) an isolated polypeptide comprising the amino acid sequence of SEQ ID NO:2, (ii) an isolated polypeptide that is the amino acid sequence of SEQ ID NO:2, and (iii) a polypeptide that is encoded by a recombinant polynucleotide comprising the polynucleotide sequence of SEQ ID NO:1. In another embodiment of this method, the inhibition comprises preventing the formation of a DXR dimer. In a further embodiment of this method, the compound forms a stable complex with the enzyme. The complex inhibits replication of, or kills, the Haemophilus influenzae carrying said enzyme. Such a contact step can occur in vitro, in vivo in an animal, particularly a mammal, infected with a Haemophilus influenzae DXR reductoisomerase enzyme or in or on a plant, or ex vivo in infected animal or plant tissue outside of the organism. Alternatively, the method may be useful as a disinfecting method, where the pathogen is present on a living or non-living surface or structure, e.g., an implant, prosthesis, skin, a plant surface, or any other surface in need of decontamination or deinfestation.

In still an additional embodiment, the invention provides a method for disinfecting a surface comprising contacting the surface with a composition comprising a compound which inhibits a DXR reductoisomerase. The surface may be a biological tissue, matrix or other substance, or a whole or part of a non-living structure, or a combination of the two. The method's contacting step comprises administering a suitable disinfecting dosage of a composition by means selected from the group consisting of coating, spraying, implanting, or soaking, among others.

Preferred antagonists of the invention and methods of modulating an activity of DXR reductoisomerase enzyme exclude fosmidomycin, derivatives thereof, FR-33289, or FR-900098, and pharmaceutical formulations of either known in the art as of the priority date of this application. Preferred antagonists and methods of modulating an activity of DXR reductoisomerase enzyme include, but are not limited to those that bind to or inhibit DXR in substantially the same way as does fosfomycin. A further embodiment of the invention is a method of modulating an activity of DXR wherein said compound is in combination with fosmidomycin, fosfomycin, FR-33289 or FR-900098. Another embodiment of the invention is a method of treatment wherein said compound is in combination with fosmidomycin, fosfomycin, FR-33289 or FR-900098. A still further embodiment of the invention is a composition comprising a compound in combination with fosmidomycin, fosfomycin, FR-33289 or FR-900098.

In other aspects of the invention are compounds or methods, each or which was (i) not known in the art prior to the filing date of this application or an application to which this application claims benefit of priority, or (ii) were not known or used by others in the United States, or patented or described in a printed publication in the United States or a foreign country, before an invention disclosed herein, or (iii) were not patented or described in a printed publication in the United States or a foreign country or in public use or on sale in the United States, more than one year prior to the filing date of this application or an application to which this application claims benefit of priority.

In yet a further preferred embodiment the compound is comprised in a moiety that binds DXR, or which compound is comprised in a moiety that binds more than one DXR homo- or hetero-dimer, or which compound binds more than one DXR homo- or hetero-dimer, or to a complex comprising a DXR and an agonist or antagonist.

In another aspect, the invention provides a pharmaceutical composition comprising a compound that inhibits or activates a Haemophilus influenzae DXR reductoisomerase enzyme in a pharmaceutically or physiologically acceptable carrier. In one embodiment, the compound is one that is identified by an assay described herein, or a compound otherwise described herein. Preferably, the composition has anti-pathogen activity, and may contain other agents and/or excipients useful in the treatment of pathogen infections.

In yet another aspect, the invention provides a pharmaceutical composition comprising a compound that inhibits or inhances biosynthesis of either menaquinone or ubiquinone as well as methods for screening for such compounds. In yet a further aspect, the invention provides a method for treating an animal, particularly a mammal or mammalian tissue infected with Haemophilus influenzae having a DXR reductoisomerase enzyme, the method comprising administering to the animal an effective amount of a pharmaceutical composition of the invention. This method involves administering the composition by any clinically desirable route, such as, but not limited to, intravenous, oral, intradermal, transdermal, intraperitoneal, intramuscular, subcutaneous, by inhalation and mucosal. Preferably this method is useful for treating such infections in a domestic animal or human, or in domestic animal or human tissue. In accordance with yet another aspect of the invention, there are provided DXR agonists and antagonists, preferably bacteriostatic or bacteriocidal agonists and antagonists. A further aspect of the invention there are provided compositions comprising a DXR polynucleotide, DXR polypeptide or an agonist or antagonists thereof for administration to a cell or to a multicellular organism.

Various changes and modifications within the spirit and scope of the disclosed invention will become readily apparent to those skilled in the art from reading the following descriptions and from reading the other parts of the present disclosure.

### DESCRIPTION OF THE INVENTION

The invention relates to DXR polypeptides and polynucleotides as described in greater detail below. In particular, the invention relates to polypeptides and polynucleotides of a DXR of H. influenzae, that is related by amino acid sequence homology to E. coli DXR polypeptide. The invention relates especially to DXR having a nucleotide and amino acid sequences set out in Table 1 as SEQ ID NO:1 and SEQ ID NO:2 respectively. Note that sequences recited in the Sequence Listing below as “DNA” represent an exemplification of the invention, since those of ordinary skill will recognize that such sequences can be usefully employed in polynucleotides in general, including ribopolynucleotides, and these are provided by the invention.

TABLE 1
DXR Polynucleotide and Polypeptide Sequences

| | |
|---|---|
| (A) H. influenzae DXR potynucleotide sequence. | |
| 5′-ATGCAAAAACAAAACATTGTCATTCTTGGTTCAACGGGATCAATCGGTAAGAGTACCCTTTCTGTTATCGAAAATAACCCTCAGAAA | [SEQ ID NO:1] |
| TATCATGCATTTGCACTCGTAGGCGGAAAAAATGTAGAAGCAATGTTTGAACAATGTATCAAATTCCGACCGCACTTTGCGGCTCTTGAT | |
| GATGTAAATGCGGCTAAAATTTTACGTGAAAAATTAATTGCGCATCATATTCCAACGGAAGTATTAGCAGGACGACGAGCTATTTGCGAA | |
| CTCGCAGCACACCCAGATGCCGATCAGATAATGGCGTCGATTGTTGGTGCAGCAGGATTGTTACCGACTCTTTCAGCGGTTAAAGCAGGT | |
| AAACGGGTATTACTGGCAAATAAAGAATCACTGGTAACCTGCGGACAGCTTTTTATTGATGCCGTAAAAAACTATGGCTCGAAGCTTTTA | |
| CCAGTAGATAGTGAACATAATGCTATCTTTCAATCATTACCGCCAGAAGCACAAGAAAAAATCGGTTTTTGCCCACTTTCTGAATTAGGT | |
| GTAAGTAAAATTATACTCACTGGTTCTGGCGGACCATTCCGTTACACGCCACTTGAACAATTCACCAACATAACACCAGAACAAGCGGTT | |
| GCACACCCTAATTGGTCTATGGGTAAAAAAATTTCTGTCGATTCAGCTACAATGATGAATAAGGGCTTGGAATACATTGAGGCTCGCTGG | |
| CTTTTCAATGCAAGTGCGGAAGAAATGGAAGTTATTATTCATCCACAATCAATTATTCATTCTATGGTACGGTATGTTGACGGCTCAGTC | |
| ATTACTCAAATGGGAAATCCAGATATGCGTACACCAATTGCAGAAACTATGGCATATCCTCACCGCACTTTTGCTGGAGTAGAACCACTC | |
| GATTTCTTTAAAATCAAAGAACTGACATTTATTGAACCTGATTTTAATCGCTATCCAAATTTAAAACTGGCTATTGATGCCTTTGCTGCG | |
| GGTCAATATGCGACAACAGCAATGAATGCAGCTAATGAAATTGCCGTACAAGCATTTTTAGATCGTCAAATTGGCTTTATGGATATTGCA | |
| AAAATTAATTCGAAAACAATTGAGAGAATTTCGCCTTATACCATTCAAAATATTGATGATGTACTCGAAATTGATGCACAAGCAAGAGAG | |
| ATTGCGAAAACACTACTTAGAGAA-3′ | |
| (B) H. influenzae DXR polypeptide sequence deduced from a polynucleotide sequence in this | |
| table. | |
| NH₂-MQKQNTVTLGSTGSTGKSTLSVIENKPQKYHAFALVGGKNVEANFEQCTKFRPHFAALDDVNAAKILREKLIAHHIPTEVLAGRRA | [SEQ ID NO:2] |
| ICELAAHPDADQIMASIVGAAGLLPTLSAVKAGKRVLLANKESLVTCGQLFTDAVKNYGSKLLPVDSEHNATFQSLPPEAQEKIGFCPLS | |
| ELGVSKTTLTGSGGPFRYTPLEQFTNTTPEQAVAHPNWSMGKKISVDSATMMNKGLEYIEARWLFNASAEEMEVITHPQSIIHSMVRYVD | |
| GSVITQMGNPDMRTPIAETMAYPHRTFAGVEPLDFFKIKELTFIEPDFNRYPNLKLAIDAFAAGQYATTAMNAANEIAVQAFLDRQIGFM | |
| DIAKINSKTIERTSPYTIQNIDDVLEIDAQAREIAKTLLRE-COOH | |

Polypeptides

DXR polypeptide of the invention is substantially phylogenetically related to other proteins of the Reductoisomerase family.

In one aspect of the invention there are provided polypeptides of H. influenzae referred to herein as “DXR” and “DXR polypeptides” as well as biologically, prophylactically, clinically or therapeutically useful variants thereof, and compositions comprising the same.

Among the particularly preferred embodiments of the invention are variants of DXR polypeptide encoded by naturally occurring alleles of a DXR gene.

The present invention further provides for an isolated polypeptide that: (a) comprises or consists of an amino acid sequence that has at least 95% identity, most preferably at least 97-99% or exact identity, to that of SEQ ID NO:2 over the entire length of SEQ ID NO:2; (b) a polypeptide encoded by an isolated polynucleotide comprising or consisting of a polynucleotide sequence that has at least 95% identity, even more preferably at least 97-99% or exact identity to SEQ ID NO:1 over the entire length of SEQ ID NO:1; (c) a polypeptide encoded by an isolated polynucleotide comprising or consisting of a polynucleotide sequence encoding a polypeptide that has at least 95% identity, even more preferably at least 97-99% or exact identity, to the amino acid sequence of SEQ ID NO:2, over the entire length of SEQ ID NO:2.

The polypeptides of the invention include a polypeptide of Table 1 [SEQ ID NO:2] (in particular a mature polypeptide) as well as polypeptides and fragments, particularly those that has a biological activity of DXR, and also those that have at least 95% identity to a polypeptide of Table 1 [SEQ ID NO:2] and also include portions of such polypeptides with such portion of the polypeptide generally comprising at least 30 amino acids and more preferably at least 50 amino acids.

The invention also includes a polypeptide consisting of or comprising a polypeptide of the formula:
(R₁)ₘ-(SEQ ID NO:2)-(R₂)ₙ
wherein each occurrence of R₁ and R₂ is independently any amino acid residue or modified amino acid residue, m is zero or is an integer between 1 and 1000, n is zero or is an integer between 1 and 1000, and SEQ ID NO:2 is an amino acid sequence of the invention. In the formula above, SEQ ID NO:2 is oriented so that its amino terminus is the amino acid residue at the left, covalently bound to R₁, and its carboxy terminus is the amino acid residue at the right, covalently bound to R₂. Any stretch of amino acid residues denoted by either R₁ or R₂, wherein m and/or n is greater than 1, may be either a heteropolymer or a homopolymer, preferably a heteropolymer. Other suitable embodiments of the invention are those wherein m is an integer between 1 and 50, 1 and 100, or 1 and 500, and n is an integer between 1 and 50, 1 and 100, or 1 and 500.

It will be appreciated by those skilled in the art, that in the above identified structure, R₁ or R₂ or both may represent sequences such as a leader or secretory sequence, a pre-, pro- or prepro-protein sequence or the like as further described below.

Fragments of the polypeptides of the invention may be employed for producing the corresponding full-length polypeptide by peptide synthesis; therefore, these variants may be employed as intermediates for producing the full-length polypeptides of the invention. The polypeptides of the present invention may be in the form of the “mature” protein or may be a part of a larger protein such as a precursor or a fusion protein. It is often advantageous to include an additional amino acid sequence that contains secretory or leader sequences, pro-sequences, sequences that aid in purification, for instance multiple histidine residues, or an additional sequence for stability during recombinant production.

Polypeptides of the present invention can be prepared in any suitable manner, for instance by isolation form naturally occurring sources, from genetically engineered host cells comprising expression systems (vide infra) or by chemical synthesis, using for instance automated peptide synthesizers, or a combination of such methods. Means for preparing such polypeptides are well understood in the art.

It is most preferred that a polypeptide of the invention is derived from H. influenzae, however, it may preferably be obtained from other organisms of the same taxonomic genus. A polypeptide of the invention may also be obtained, for example, from organisms of the same taxonomic family or order.

A fragment is a variant polypeptide having an amino acid sequence that is entirely the same as part but not all of any amino acid sequence of any polypeptide of the invention. As with DXR polypeptides, fragments may be “free-standing,” or comprised within a larger polypeptide of which they form a part or region, most preferably as a single continuous region in a single larger polypeptide.

Preferred fragments include, for example, truncation polypeptides having a portion of an amino acid sequence of Table 1 [SEQ ID NO:2], or of variants thereof, such as a continuous series of residues that includes an amino- and/or carboxyl-terminal amino acid sequence. Degradation forms of the polypeptides of the invention produced by or in a host cell, particularly a H. influenzae, are also preferred. Further preferred are fragments characterized by structural or functional attributes such as fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, substrate binding region, and high antigenic index regions.

Further preferred fragments include an isolated polypeptide comprising an amino acid sequence having at least 15, 20, 30, 40, 50 or 100 contiguous amino acids from the amino acid sequence of SEQ ID NO:2, or an isolated polypeptide comprising an amino acid sequence having at least 15, 20, 30, 40, 50 or 100 contiguous amino acids truncated or deleted from the amino acid sequence of SEQ ID NO:2.

Fragments of the polypeptides of the invention may be employed for producing the corresponding full-length polypeptide by peptide synthesis; therefore, these variants may be employed as intermediates for producing the full-length polypeptides of the invention.

Polynucleotides

It is an object of the invention to provide polynucleotides that encode DXR polypeptides, particularly polynucleotides that encode a polypeptide herein designated DXR.

In a particularly preferred embodiment of the invention the polynucleotide comprises a region encoding DXR polypeptides comprising a sequence set out in Table 1 [SEQ ID NO:1] that includes a full length gene, or a variant thereof. This invention provides that this full length gene is essential to the growth and/or survival of an organism that possesses it, such as H. influenzae.

As a further aspect of the invention there are provided isolated nucleic acid molecules encoding and/or expressing DXR polypeptides and polynucleotides, particularly H. influenzae DXR polypeptides and polynucleotides, including, for example, unprocessed RNAs, ribozyme RNAs, mRNAs, cDNAs, genomic DNAs, B- and Z-DNAs. Further embodiments of the invention include biologically, prophylactically, clinically or therapeutically useful polynucleotides and polypeptides, and variants thereof, and compositions comprising the same.

Another aspect of the invention relates to isolated polynucleotides, including at least one full length gene, that encodes a DXR polypeptide having a deduced amino acid sequence of Table 1 [SEQ ID NO:2] and polynucleotides closely related thereto and variants thereof.

In another particularly preferred embodiment of the invention there is a DXR polypeptide from H. influenzae comprising or consisting of an amino acid sequence of Table 1 [SEQ ID NO:2], or a variant thereof.

Using the information provided herein, such as a polynucleotide sequence set out in Table 1 [SEQ ID NO:1], a polynucleotide of the invention encoding DXR polypeptide may be obtained using standard cloning and screening methods, such as those for cloning and sequencing chromosomal DNA fragments from bacteria using H. influenzae cells as starting material, followed by obtaining a full length clone. For example, to obtain a polynucleotide sequence of the invention, such as a polynucleotide sequence given in Table 1 [SEQ ID NO:1], typically a library of clones of chromosomal DNA of H. influenzae, in E. coli or some other suitable host is probed with a radiolabeled oligonucleotide, preferably a 17-mer or longer, derived from a partial sequence. Clones carrying DNA identical to that of the probe can then be distinguished using stringent hybridization conditions. By sequencing the individual clones thus identified by hybridization with sequencing primers designed from the original polypeptide or polynucleotide sequence it is then possible to extend the polynucleotide sequence in both directions to determine a full length gene sequence. Conveniently, such sequencing is performed, for example, using denatured double stranded DNA prepared from a plasmid clone. Suitable techniques are described by Maniatis, T., Fritsch, E. F. and Sambrook et al., MOLECULAR CLONING, A LABORATORY MANUAL, 2nd Ed.; Cold Spring Harbor Laboratory Press, Cold Spring Harbor, N.Y. (1989). (see in particular Screening By Hybridization 1.90 and Sequencing Denatured Double-Stranded DNA Templates 13.70). Direct genomic DNA sequencing may also be performed to obtain a full length gene sequence. Illustrative of the invention, each polynucleotide set out in Table 1 [SEQ ID NO:1] was discovered in a DNA library derived from H. influenzae.

Moreover, each DNA sequence set out in Table 1 [SEQ ID NO: I] contains an open reading frame encoding a protein having about the number of amino acid residues set forth in Table 1 [SEQ ID NO:2] with a deduced molecular weight that can be calculated using amino acid residue molecular weight values well known to those skilled in the art. The polynucleotide of SEQ ID NO:1, between nucleotide number ATG and the stop codon that begins at nucleotide number TAG of SEQ ID NO:1, encodes the polypeptide of SEQ ID NO:2.

In a further aspect, the present invention provides for an isolated polynucleotide comprising or consisting of: (a) a polynucleotide sequence that has at least 95% identity, even more preferably at least 97-99% or exact identity to SEQ ID NO:1 over the entire length of SEQ ID NO:1; (b) a polynucleotide sequence encoding a polypeptide that has at least 95% identity, even more preferably at least 97-99% or 100% exact, to the amino acid sequence of SEQ ID NO:2, over the entire length of SEQ ID NO:2.

A polynucleotide encoding a polypeptide of the present invention, including homologs and orthologs from species other than H. influenzae, may be obtained by a process that comprises the steps of screening an appropriate library under stringent hybridization conditions with a labeled or detectable probe consisting of or comprising the sequence of SEQ ID NO:1 or a fragment thereof; and isolating a full-length gene and/or genomic clones comprising said polynucleotide sequence. The invention provides a polynucleotide sequence identical over its entire length to a coding sequence (open reading frame) in Table 1 [SEQ ID NO:1]. Also provided by the invention is a coding sequence for a mature polypeptide or a fragment thereof, by itself as well as a coding sequence for a mature polypeptide or a fragment in reading frame with another coding sequence, such as a sequence encoding a leader or secretory sequence, a pre-, or pro- or prepro-protein sequence. The polynucleotide of the invention may also comprise at least one non-coding sequence, including for example, but not limited to at least one non-coding 5′ and 3′ sequence, such as the transcribed but non-translated sequences, termination signals (such as rho-dependent and rho-independent termination signals), ribosome binding sites, Kozak sequences, sequences that stabilize mRNA, introns, and polyadenylation signals. The polynucleotide sequence may also comprise additional coding sequence encoding additional amino acids. For example, a marker sequence that facilitates purification of a fused polypeptide can be encoded. In certain embodiments of the invention, the marker sequence is a hexa-histidine peptide, as provided in the pQE vector (Qiagen, Inc., Hibden, Germany) and described in Gentz et al., Proc. Natl. Acad. Sci., USA 86: 821-824 (1989), or an HA peptide tag (Wilson et al., Cell 37: 767 (1984), both of that may be useful in purifying polypeptide sequence fused to them. Polynucleotides of the invention also include, but are not limited to, polynucleotides comprising a structural gene and its naturally associated sequences that control gene expression.

A preferred embodiment of the invention is a polynucleotide of, consisting of, or comprising nucleotide ATG to the nucleotide immediately upstream of or including nucleotide TAG set forth in SEQ ID NO:1 of Table 1, both of that encode a DXR polypeptide.

The invention also includes a polynucleotide consisting of or comprising a polynucleotide of the formula:
(R₁)ₘ-(SEQ ID NO:1)-(R₂)ₙ
wherein each occurrence of R₁ and R₂ is independently any nucleic acid residue or modified nucleic acid residue, m is zero or an integer between 1 and 3000, n is zero or an integer between 1 and 3000, and SEQ ID NO:1 is a nucleotide sequence of the invention. In the polynucleotide formula above, SEQ ID NO:1 is oriented so that its 5′ end nucleic acid residue is at the left, bound to R₁, and its 3′ end nucleic acid residue is at the right, bound to R₂. Any stretch of nucleic acid residues denoted by R₁ or R₂, wherein m or n or both are greater than 1, may be either a heteropolymer or a homopolymer, preferably a heteropolymer. Where R₁ and R₂ are joined together by a covalent bond, the polynucleotide of the above formula is a closed, circular polynucleotide, that can be a double-stranded polynucleotide wherein the formula shows a first strand to which the second strand is complementary. In another embodiment m or n or both are an integer between 1 and 1000 or zero. Other embodiments of the invention include those wherein m is an integer between 1 and 50, 1 and 100 or 1 and 500, and n is an integer between 1 and 50, 1 and 100, or 1 and 500.

It is most preferred that a polynucleotide of the invention is derived from H. influenzae, however, it may preferably be obtained from other organisms of the same taxonomic genus. A polynucleotide of the invention may also be obtained, for example, from organisms of the same taxonomic family or order.

The term “polynucleotide encoding a polypeptide” as used herein encompasses polynucleotides that include a sequence encoding a polypeptide of the invention, particularly a bacterial polypeptide and more particularly a polypeptide of the H. influenzae DXR having an amino acid sequence set out in Table 1 [SEQ ID NO:2]. The term also encompasses polynucleotides that include a single continuous region or discontinuous regions encoding the polypeptide (for example, polynucleotides interrupted by integrated phage, an integrated insertion sequence, an integrated vector sequence, an integrated transposon sequence, or due to RNA editing or genomic DNA reorganization) together with additional regions, that also may comprise coding and/or non-coding sequences.

The invention further relates to variants of the polynucleotides described herein that encode variants of a polypeptide having a deduced amino acid sequence of Table 1 [SEQ ID NO:2]. Fragments of polynucleotides of the invention may be used, for example, to synthesize full-length polynucleotides of the invention.

Further particularly preferred embodiments are polynucleotides encoding DXR variants, that have the amino acid sequence of DXR polypeptide of Table 1 [SEQ ID NO:2] in which several, a few, 5 to 10, 1 to 5, 1 to 3, 2, 1 or no amino acid residues are substituted, modified, deleted and/or added, in any combination. Especially preferred among these are silent substitutions, additions and deletions, that do not alter the properties and activities of DXR polypeptide.

Preferred isolated polynucleotide embodiments also include polynucleotide fragments, such as a polynucleotide comprising a nucleic acid sequence having at least 15, 20, 30, 40, 50 or 100 contiguous nucleic acids from the polynucleotide sequence of SEQ ID NO:1, or an polynucleotide comprising a nucleic acid sequence having at least 15, 20, 30, 40, 50 or 100 contiguous nucleic acids truncated or deleted from the 5′ and/or 3′ end of the polynucleotide sequence of SEQ ID NO:1.

Further preferred embodiments of the invention are polynucleotides that are at least 95% or 97% identical over their entire length to a polynucleotide encoding DXR polypeptide having an amino acid sequence set out in Table 1 [SEQ ID NO:2], and polynucleotides that are complementary to such polynucleotides. Most highly preferred are polynucleotides that comprise a region that is at least 95% are especially preferred. Furthermore, those with at least 97% are highly preferred among those with at least 95%, and among these those with at least 98% and at least 99% are particularly highly preferred, with at least 99% being the more preferred.

Preferred embodiments are polynucleotides encoding polypeptides that retain substantially the same biological function or activity as a mature polypeptide encoded by a DNA of Table 1 [SEQ ID NO:1].

In accordance with certain preferred embodiments of this invention there are provided polynucleotides that hybridize, particularly under stringent conditions, to DXR polynucleotide sequences, such as those polynucleotides in Table 1.

The invention further relates to polynucleotides that hybridize to the polynucleotide sequences provided herein. In this regard, the invention especially relates to polynucleotides that hybridize under stringent conditions to the polynucleotides described herein. A specific example of stringent hybridization conditions is overnight incubation at 42° C. in a solution comprising: 50% formamide, 5×SSC (150 mM NaCl, 15 mM trisodium citrate), 50 mM sodium phosphate (pH7.6), 5× Denhardt's solution, 10% dextran sulfate, and 20 micrograms/ml of denatured, sheared salmon sperm DNA, followed by washing the hybridization support in 0.1×SSC at about 65° C. Hybridization and wash conditions are well known and exemplified in Sambrook, et al., Molecular Cloning: A Laboratory Manual, Second Edition, Cold Spring Harbor, N.Y., (1989), particularly Chapter 11 therein. Solution hybridization may also be used with the polynucleotide sequences provided by the invention.

The invention also provides a polynucleotide consisting of or comprising a polynucleotide sequence obtained by screening an appropriate library comprising a complete gene for a polynucleotide sequence set forth in SEQ ID NO:1 under stringent hybridization conditions with a probe having the sequence of said polynucleotide sequence set forth in SEQ ID NO:1 or a fragment thereof, and isolating said polynucleotide sequence. Fragments useful for obtaining such a polynucleotide include, for example, probes and primers fully described elsewhere herein.

As discussed elsewhere herein regarding polynucleotide assays of the invention, for instance, the polynucleotides of the invention, may be used as a hybridization probe for RNA, cDNA and genomic DNA to isolate full-length cDNAs and genomic clones encoding DXR and to isolate cDNA and genomic clones of other genes that have a high identity, particularly high sequence identity, to a DXR gene. Such probes generally will comprise at least 15 nucleotide residues or base pairs. Preferably, such probes will have at least 30 nucleotide residues or base pairs and may have at least 50 nucleotide residues or base pairs. Particularly preferred probes will have at least 20 nucleotide residues or base pairs and will have lee than 30 nucleotide residues or base pairs.

A coding region of a DXR gene may be isolated by screening using a DNA sequence provided in Table 1 [SEQ ID NO:1] to synthesize an oligonucleotide probe. A labeled oligonucleotide having a sequence complementary to that of a gene of the invention is then used to screen a library of cDNA, genomic DNA or mRNA to determine which members of the library the probe hybridizes to.

There are several methods available and well known to those skilled in the art to obtain full-length DNAs, or extend short DNAs, for example those based on the method of Rapid Amplification of cDNA ends (RACE) (see, for example, Frohman, et al., PNAS USA 85: 8998-9002, 1988). Recent modifications of the technique, exemplified by the Marathon™ technology (Clontech Laboratories Inc.) for example, have significantly simplified the search for longer cDNAs. In the Marathon™ technology, cDNAs have been prepared from mRNA extracted from a chosen tissue and an ‘adaptor’ sequence ligated onto each end. Nucleic acid amplification (PCR) is then carried out to amplify the “missing” 5′ end of the DNA using a combination of gene specific and adaptor specific oligonucleotide primers. The PCR reaction is then repeated using “nested” primers, that is, primers designed to anneal within the amplified product (typically an adaptor specific primer that anneals further 3′ in the adaptor sequence and a gene specific primer that anneals further 5′ in the selected gene sequence). The products of this reaction can then be analyzed by DNA sequencing and a full-length DNA constructed either by joining the product directly to the existing DNA to give a complete sequence, or carrying out a separate full-length PCR using the new sequence information for the design of the 5′ primer.

The polynucleotides and polypeptides of the invention may be employed, for example, as research reagents and materials for discovery of treatments for diseases, particularly human diseases, as further discussed herein relating to polynucleotide assays.

The polynucleotides of the invention that are oligonucleotides derived from a sequence of Table 1 [SEQ ID NOS:1 or 2] may be used in the processes herein as described, but preferably for PCR, to determine whether or not the polynucleotides identified herein in whole or in part are transcribed in bacteria in infected tissue.

The invention also provides polynucleotides that encode a polypeptide that is a mature protein plus additional amino or carboxyl-terminal amino acids, or amino acids interior to a mature polypeptide (when a mature form has more than one polypeptide chain, for instance). Such sequences may play a role in processing of a protein from precursor to a mature form, may allow protein transport, may lengthen or shorten protein half-life or may facilitate manipulation of a protein for assay or production, among other things. As generally is the case in vivo, the additional amino acids may be processed away from a mature protein by cellular enzymes.

For each and every polynucleotide of the invention there is provided a polynucleotide complementary to it. It is preferred that these complementary polynucleotides are fully complementary to each polynucleotide with which they are complementary.

A precursor protein, having a mature form of the polypeptide fused to one or more prosequences may be an inactive form of the polypeptide. When prosequences are removed such inactive precursors generally are activated. Some or all of the prosequences may be removed before activation. Generally, such precursors are called proproteins.

As will be recognized, the entire polypeptide encoded by an open reading frame is often not required for activity. Accordingly, it has become routine in molecular biology to map the boundaries of the primary structure required for activity with N-terminal and C-terminal deletion experiments. These experiments utilize exonuclease digestion or convenient restriction sites to cleave coding nucleic acid sequence. For example, Promega (Madison, Wis.) sell an Erase-a-base™ system that uses Exonuclease III designed to facilitate analysis of the deletion products (protocol available at www.promega.com). The digested endpoints can be repaired (e.g., by ligation to synthetic linkers) to the extent necessary to preserve an open reading frame. In this way, the nucleic acid of SEQ ID NO:1 readily provides contiguous fragments of SEQ ID NO:2 sufficient to provide an activity, such as an enzymatic, binding or antibody-inducing activity. Nucleic acid sequences encoding such fragments of SEQ ID NO:2 and variants thereof as described herein are within the invention, as are polypeptides so encoded.

As is known in the art, portions of the N-terminal and/or C-terminal sequence of a protein can generally be removed without serious consequence to the function of the protein. The amount of sequence that can be removed is often quite substantial. The nucleic acid cutting and deletion methods used for creating such deletion variants are now quite routine. Accordingly, any contiguous fragment of SEQ ID NO:2 which retains at least 20%, preferably at least 50%, of an activity of the polypeptide encoded by the gene for SEQ ID NO:2 is within the invention, as are corresponding fragment which are 70%, 80%, 90%, 95%, 97%, 98% or 99% identical to such contiguous fragments. In one embodiment, the contiguous fragment comprises at least 70% of the amino acid residues of SEQ ID NO:2, preferably at least 80%, 90% or 95% of the residues.

In sum, a polynucleotide of the invention may encode a mature protein, a mature protein plus a leader sequence (that may be referred to as a preprotein), a precursor of a mature protein having one or more prosequences that are not the leader sequences of a preprotein, or a preproprotein, that is a precursor to a proprotein, having a leader sequence and one or more prosequences, that generally are removed during processing steps that produce active and mature forms of the polypeptide.

Vectors, Host Cells, Expression Systems

The invention also relates to vectors that comprise a polynucleotide or polynucleotides of the invention, host cells that are genetically engineered with vectors of the invention and the production of polypeptides of the invention by recombinant techniques. Cell-free translation systems can also be employed to produce such proteins using RNAs derived from the DNA constructs of the invention.

Recombinant polypeptides of the present invention may be prepared by processes well known in those skilled in the art from genetically engineered host cells comprising expression systems. Accordingly, in a further aspect, the present invention relates to expression systems that comprise a polynucleotide or polynucleotides of the present invention, to host cells that are genetically engineered with such expression systems, and to the production of polypeptides of the invention by recombinant techniques.

For recombinant production of the polypeptides of the invention, host cells can be genetically engineered to incorporate expression systems or portions thereof or polynucleotides of the invention. Introduction of a polynucleotide into the host cell can be effected by methods described in many standard laboratory manuals, such as Davis, et al., BASIC METHODS IN MOLECULAR BIOLOGY, (1986) and Sambrook, et al., MOLECULAR CLONING: A LABORATORY MANUAL, 2nd Ed., Cold Spring Harbor Laboratory Press, Cold Spring Harbor, N.Y. (1989), such as, calcium phosphate transfection, DEAE-dextran mediated transfection, transvection, microinjection, cationic lipid-mediated transfection, electroporation, transduction, scrape loading, ballistic introduction and infection.

Representative examples of appropriate hosts include bacterial cells, such as cells of streptococci, staphylococci, enterococci E. coli, streptomyces, cyanobacteria, Bacillus subtilis, and H. influenzae; fungal cells, such as cells of a yeast, Kluveromyces, Saccharomyces, a basidiomycete, Candida albicans and Aspergillus; insect cells such as cells of Drosophila S2 and Spodoptera Sf9; animal cells such as CHO, COS, HeLa, C127, 3T3, BHK, 293, CV-1 and Bowes melanoma cells; and plant cells, such as cells of a gymnosperm or angiosperm.

A great variety of expression systems can be used to produce the polypeptides of the invention. Such vectors include, among others, chromosomal-, episomal- and virus-derived vectors, for example, vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses, picomaviruses and retroviruses, and vectors derived from combinations thereof, such as those derived from plasmid and bacteriophage genetic elements, such as cosmids and phagemids. The expression system constructs may comprise control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al., MOLECULAR CLONING, A LABORATORY MANUAL, (supra).

In recombinant expression systems in eukaryotes, for secretion of a translated protein into the lumen of the endoplasmic reticulum, into the periplasmic space or into the extracellular environment, appropriate secretion signals may be incorporated into the expressed polypeptide. These signals may be endogenous to the polypeptide or they may be heterologous signals.

Polypeptides of the invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography. Most preferably, high performance liquid chromatography is employed for purification. Well known techniques for refolding protein may be employed to regenerate active conformation when the polypeptide is denatured during isolation and or purification.

Antagonists and Agonists—Assays and Molecules

Polypeptides and polynucleotides of the invention may also be used to assess the binding of small molecule substrates and ligands in, for example, cells, cell-free preparations, chemical libraries, and natural product mixtures. These substrates and ligands may be natural substrates and ligands or may be structural or functional mimetics. See, e.g., Coligan et al., Current Protocols in Immunology 1(2): Chapter 5 (1991).

Polypeptides and polynucleotides of the present invention are responsible for many biological functions, including many disease states, in particular the Diseases herein mentioned. It is therefore desirable to devise screening methods to identify compounds that agonize (e.g., stimulate) or that antagonize (e.g., inhibit) the function of the polypeptide or polynucleotide. Accordingly, in a further aspect, the present invention provides for a method of screening compounds to identify those that agonize or that antagonize the function of a polypeptide or polynucleotide of the invention, as well as related polypeptides and polynucleotides. In general, agonists or antagonists (e.g., inhibitors) may be employed for therapeutic and prophylactic purposes for such Diseases as herein mentioned. Compounds may be identified from a variety of sources, for example, cells, cell-free preparations, chemical libraries, and natural product mixtures. Such agonists and antagonists so-identified may be natural or modified substrates, ligands, receptors, enzymes, etc., as the case may be, of DXR polypeptides and polynucleotides; or may be structural or functional mimetics thereof (see Coligan et al., Current Protocols in Immunology 1(2): Chapter 5 (1991)).

The screening methods may simply measure the binding of a candidate compound to the polypeptide or polynucleotide, or to cells or membranes bearing the polypeptide or polynucleotide, or a fusion protein of the polypeptide by means of a label directly or indirectly associated with the candidate compound. Alternatively, the screening method may involve competition with a labeled competitor. Further, these screening methods may test whether the candidate compound results in a signal generated by activation or inhibition of the polypeptide or polynucleotide, using detection systems appropriate to the cells comprising the polypeptide or polynucleotide. Inhibitors of activation are generally assayed in the presence of a known agonist and the effect on activation by the agonist by the presence of the candidate compound is observed. Constitutively active polypeptide and/or constitutively expressed polypeptides and polynucleotides may be employed in screening methods for inverse agonists, in the absence of an agonist or antagonist, by testing whether the candidate compound results in inhibition of activation of the polypeptide or polynucleotide, as the case may be. Further, the screening methods may simply comprise the steps of mixing a candidate compound with a solution comprising a polypeptide or polynucleotide of the present invention, to form a mixture, measuring DXR polypeptide and/or polynucleotide activity in the mixture, and comparing the DXR polypeptide and/or polynucleotide activity of the mixture to a standard. Fusion proteins, such as those made from Fc portion and DXR polypeptide, as herein described, can also be used for high-throughput screening assays to identify antagonists of the polypeptide of the present invention, as well as of phylogenetically and and/or functionally related polypeptides (see D. Bennett et al., J Mol Recognition, 8:52-58 (1995); and K. Johanson et al., J Biol Chem, 270(16): 9459-9471 (1995)).

Preferred antagonists of the invention exclude fosmidomycin and derivatives thereof known in the art as of the priority date of this application. Preferred antagonists include those that bind to or inhibit DXR in substantially the same way as does fosfomycin.

The polynucleotides, polypeptides and antibodies that bind to and/or interact with a polypeptide of the present invention may also be used to configure screening methods for detecting the effect of added compounds on the production of mRNA and/or polypeptide in cells. For example, an ELISA assay may be constructed for measuring secreted or cell associated levels of polypeptide using monoclonal and polyclonal antibodies by standard methods known in the art. This can be used to discover agents that may inhibit or enhance the production of polypeptide (also called antagonist or agonist, respectively) from suitably manipulated cells or tissues.

The invention also provides a method of screening compounds to identify those that enhance (agonist) or block (antagonist) the action of DXR polypeptides or polynucleotides, particularly those compounds that are bacteristatic and/or bactericidal. The method of screening may involve high-throughput techniques. For example, to screen for agonists or antagonists, a synthetic reaction mix, a cellular compartment, such as a membrane, cell envelope or cell wall, or a preparation of any thereof, comprising DXR polypeptide and a labeled substrate or ligand of such polypeptide is incubated in the absence or the presence of a candidate molecule that may be a DXR agonist or antagonist. The ability of the candidate molecule to agonize or antagonize the DXR polypeptide is reflected in decreased binding of the labeled ligand or decreased production of product from such substrate. Molecules that bind gratuitously, i.e., without inducing the effects of DXR polypeptide are most likely to be good antagonists. Molecules that bind well and, as the case may be, increase the rate of product production from substrate, increase signal transduction, or increase chemical channel activity are agonists. Detection of the rate or level of, as the case may be, production of product from substrate, signal transduction, or chemical channel activity may be enhanced by using a reporter system. Reporter systems that may be useful in this regard include but are not limited to colorimetric, labeled substrate converted into product, a reporter gene that is responsive to changes in DXR polynucleotide or polypeptide activity, and binding assays known in the art. Polypeptides of the invention may be used to identify membrane bound or soluble receptors, if any, for such polypeptide, through standard receptor binding techniques known in the art. These techniques include, but are not limited to, ligand binding and crosslinking assays in which the polypeptide is labeled with a radioactive isotope (for instance, ¹²⁵I), chemically modified (for instance, biotinylated), or fused to a peptide sequence suitable for detection or purification, and incubated with a source of the putative receptor (e.g., cells, cell membranes, cell supernatants, tissue extracts, bodily materials). Other methods include biophysical techniques such as surface plasmon resonance and spectroscopy. These screening methods may also be used to identify agonists and antagonists of the polypeptide that compete with the binding of the polypeptide to its receptor(s), if any. Standard methods for conducting such assays are well understood in the art.

The fluorescence polarization value for a fluorescently-tagged molecule depends on the rotational correlation time or tumbling rate. Protein complexes, such as formed by DXR polypeptide associating with another DXR polypeptide or other polypeptide, labeled to comprise a fluorescently-labeled molecule will have higher polarization values than a fluorescently labeled monomeric protein. It is preferred that this method be used to characterize small molecules that disrupt polypeptide complexes.

Fluorescence energy transfer may also be used characterize small molecules that interfere with the formation of DXR polypeptide dimers, trimers, tetramers or higher order structures, or structures formed by DXR polypeptide bound to another polypeptide. DXR polypeptide can be labeled with both a donor and acceptor fluorophore. Upon mixing of the two labeled species and excitation of the donor fluorophore, fluorescence energy transfer can be detected by observing fluorescence of the acceptor. Compounds that block dimerization will inhibit fluorescence energy transfer.

Surface plasmon resonance can be used to monitor the effect of small molecules on DXR polypeptide self-association as well as an association of DXR polypeptide and another polypeptide or small molecule. DXR polypeptide can be coupled to a sensor chip at low site density such that covalently bound molecules will be monomeric. Solution protein can then passed over the DXR polypeptide-coated surface and specific binding can be detected in real-time by monitoring the change in resonance angle caused by a change in local refractive index. This technique can be used to characterize the effect of small molecules on kinetic rates and equilibrium binding constants for DXR polypeptide self-association as well as an association of DXR polypeptide and another polypeptide or small molecule.

A scintillation proximity assay may be used to characterize the interaction between an association of DXR polypeptide with another DXR polypeptide or a different polypeptide. DXR polypeptide can be coupled to a scintillation-filled bead. Addition of radio-labeled DXR polypeptide results in binding where the radioactive source molecule is in close proximity to the scintillation fluid. Thus, signal is emitted upon DXR polypeptide binding and compounds that prevent DXR polypeptide self-association or an association of DXR polypeptide and another polypeptide or small molecule will diminish signal.

In other embodiments of the invention there are provided methods for identifying compounds that bind to or otherwise interact with and inhibit or activate an activity or expression of a polypeptide and/or polynucleotide of the invention comprising: contacting a polypeptide and/or polynucleotide of the invention with a compound to be screened under conditions to permit binding to or other interaction between the compound and the polypeptide and/or polynucleotide to assess the binding to or other interaction with the compound, such binding or interaction preferably being associated with a second component capable of providing a detectable signal in response to the binding or interaction of the polypeptide and/or polynucleotide with the compound; and determining whether the compound binds to or otherwise interacts with and activates or inhibits an activity or expression of the polypeptide and/or polynucleotide by detecting the presence or absence of a signal generated from the binding or interaction of the compound with the polypeptide and/or polynucleotide.

Another example of an assay for DXR agonists is a competitive assay that combines DXR and a potential agonist with DXR-binding molecules, recombinant DXR binding molecules, natural substrates or ligands, or substrate or ligand mimetics, under appropriate conditions for a competitive inhibition assay. DXR can be labeled, such as by radioactivity or a colorimetric compound, such that the number of DXR molecules bound to a binding molecule or converted to product can be determined accurately to assess the effectiveness of the potential antagonist.

It will be readily appreciated by the skilled artisan that a polypeptide and/or polynucleotide of the present invention may also be used in a method for the structure-based design of an agonist or antagonist of the polypeptide and/or polynucleotide, by: (a) determining in the first instance the three-dimensional structure of the polypeptide and/or polynucleotide, or complexes thereof; (b) deducing the three-dimensional structure for the likely reactive site(s), binding site(s) or motif(s) of an agonist or antagonist; (c) synthesizing candidate compounds that are predicted to bind to or react with the deduced binding site(s), reactive site(s), and/or motif(s); and
(d) testing whether the candidate compounds are indeed agonists or antagonists.

It will be further appreciated that this will normally be an iterative process, and this iterative process may be performed using automated and computer-controlled steps.

In a further aspect, the present invention provides methods of treating abnormal conditions such as, for instance, a Disease, related to either an excess of, an under-expression of, an elevated activity of, or a decreased activity of DXR polypeptide and/or polynucleotide.

If the expression and/or activity of the polypeptide and/or polynucleotide is in excess, several approaches are available. One approach comprises administering to an individual in need thereof an inhibitor compound (antagonist) as herein described, optionally in combination with a pharmaceutically acceptable carrier, in an amount effective to inhibit the function and/or expression of the polypeptide and/or polynucleotide, such as, for example, by blocking the binding of ligands, substrates, receptors, enzymes, etc., or by inhibiting a second signal, and thereby alleviating the abnormal condition. In another approach, soluble forms of the polypeptides still capable of binding the ligand, substrate, enzymes, receptors, etc. in competition with endogenous polypeptide and/or polynucleotide may be administered. Typical examples of such competitors include fragments of the DXR polypeptide and/or polypeptide.

In still another approach, expression of the gene encoding endogenous DXR polypeptide can be inhibited using expression blocking techniques. This blocking may be targeted against any step in gene expression, but is preferably targeted against transcription and/or translation. An examples of a known technique of this sort involve the use of antisense sequences, either internally generated or separately administered (see, for example, O'Connor, J Neurochem (1991) 56:560 in Oligodeoxynucleotides as Antisense Inhibitors of Gene Expression, CRC Press, Boca Raton, Fla. (1988)). Alternatively, oligonucleotides that form triple helices with the gene can be supplied (see, for example, Lee et al., Nucleic Acids Res (1979) 6:3073; Cooney et al., Science (1988) 241:456; Dervan et al., Science (1991) 251:1360). These oligomers can be administered per se or the relevant oligomers can be expressed in vivo.

Each of the polynucleotide sequences provided herein may be used in the discovery and development of antibacterial compounds. The encoded protein, upon expression, can be used as a target for the screening of antibacterial drugs. Additionally, the polynucleotide sequences encoding the amino terminal regions of the encoded protein or Shine-Delgarno or other translation facilitating sequences of the respective mRNA can be used to construct antisense sequences to control the expression of the coding sequence of interest.

The invention also provides the use of the polypeptide, polynucleotide, agonist or antagonist of the invention to interfere with the initial physical interaction between a pathogen or pathogens and an eukaryotic, preferably mammalian, host responsible for sequelae of infection. In particular, the molecules of the invention may be used: in the prevention of adhesion of bacteria, in particular gram positive and/or gram negative bacteria, to eukaryotic, preferably mammalian, extracellular matrix proteins on in-dwelling devices or to extracellular matrix proteins in wounds; to block bacterial adhesion between eukaryotic, preferably mammalian, extracellular matrix proteins and bacterial DXR proteins that mediate tissue damage and/or; to block the normal progression of pathogenesis in infections initiated other than by the implantation of in-dwelling devices or by other surgical techniques.

In accordance with yet another aspect of the invention, there are provided DXR agonists and antagonists, preferably bacteristatic or bactericidal agonists and antagonists.

The antagonists and agonists of the invention may be employed, for instance, to prevent, inhibit and/or treat diseases.

Antagonists of the invention include, among others, small organic molecules, peptides, polypeptides and antibodies that bind to a polynucleotide and/or polypeptide of the invention and thereby inhibit or extinguish its activity or expression. Antagonists also may be small organic molecules, a peptide, a polypeptide such as a closely related protein or antibody that binds the same sites on a binding molecule, such as a binding molecule, without inducing DXR-induced activities, thereby preventing the action or expression of DXR polypeptides and/or polynucleotides by excluding DXR polypeptides and/or polynucleotides from binding.

Antagonists of the invention also include a small molecule that binds to and occupies the binding site of the polypeptide thereby preventing binding to cellular binding molecules, such that normal biological activity is prevented. Examples of small molecules include but are not limited to small organic molecules, peptides or peptide-like molecules. Other antagonists include antisense molecules (see Okano, J. Neurochem. 56: 560 (1991); OLIGODEOXYNUCLEOTIDES AS ANTISENSE INHIBITORS OF GENE EXPRESSION, CRC Press, Boca Raton, Fla. (1988), for a description of these molecules). Preferred antagonists include compounds related to and variants of DXR.

Other examples of polypeptide antagonists include antibodies or, in some cases, oligonucleotides or proteins that are closely related to the ligands, substrates, receptors, enzymes, etc., as the case may be, of the polypeptide, e.g., a fragment of the ligands, substrates, receptors, enzymes, etc.; or small molecules that bind to the polypeptide of the present invention but do not elicit a response, so that an activity of the polypeptide is prevented.

Small molecules of the invention preferably have a molecular weight below 2,000 daltons, more preferably between 300 and 1,000 daltons, and most preferably between 400 and 700 daltons. It is preferred that these small molecules are organic molecules.

Helicobacter pylori (herein “H pylori”) bacteria infect the stomachs of over one-third of the world's population causing stomach cancer, ulcers, and gastritis (International Agency for Research on Cancer (1994) Schistosomes, Liver Flukes and Helicobacter Pylori (International Agency for Research on Cancer, Lyon, France, http://www.uicc.ch/ecp/ecp2904.htm). Moreover, the International Agency for Research on Cancer recently recognized a cause-and-effect relationship between H. pylori and gastric adenocarcinoma, classifying the bacterium as a Group I (definite) carcinogen. Preferred antimicrobial compounds of the invention (agonists and antagonists of DXR polypeptides and/or polynucleotides) found using screens provided by the invention, or known in the art, particularly narrow-spectrum antibiotics, should be useful in the treatment of H. pylori infection. Such treatment should decrease the advent of H. pylori-induced cancers, such as gastrointestinal carcinoma. Such treatment should also prevent, inhibit and/or cure gastric ulcers and gastritis.

Pharmaceutical Compositions

Pharmaceutical compositions of this invention are designed to treat infection of an infected mammal, e.g., human by Haemophilus influenzae possessing a DXR reductoisomerase. It is preferred in the invention that the methods of treatment or modulation of a DXR activity herein are used with Haemophilus influenzae as the pathogen, or other Gram negative bacteria, but preferably exclude E. coli and multicellular plants. A compound or mixture of compounds of the present invention may be formulated into an antipathogen composition with a pharmaceutically acceptable carrier and other optional components. For use in such compositions, a selected compound may be produced preferably synthetically, but also recombinantly, as disclosed herein, or by a known method.

The compounds may be employed in pharmaceutical compositions individually. Alternatively, for the purposes of enhancing pharmacokinetics or bioavailability without eliciting immune responses, one or more compounds may be fused or conjugated to other moieties, e.g., carrier proteins or other chemical moieties to enhance stability or delivery, to improve the production, or to change an activity spectrum of the compound. As a few well-known examples, such moieties may be human albumin, polyethylene glycol, biopolymers or other naturally or non-naturally occurring polymers, as well as other materials. In one embodiment, the moiety is desirably a molecule which can enhance the stability of the compound. One of skill in the art can readily select an appropriate conjugation moiety. For certain of the same purposes, one or more of the compounds may be designed as a synthetic compound fused to a carrier protein or other molecule. Still alternatively multiple of a compound provided herein may be combined in a multi-part compound composition. Any compounds of this multi-part composition may be coupled to the same carrier, or different compounds may be coupled individually as compounds to the same or a different immunologically inert carrier proteins.

As pharmaceutical compositions, these compositions are admixed with a pharmaceutically acceptable vehicle or carrier suitable for administration. These compounds may be combined in a single pharmaceutical preparation for administration. Suitable pharmaceutically acceptable or physiologically acceptable carriers for use in a pharmaceutical composition of the invention are well known to those of skill in the art. Such carriers include, for example, saline, buffered saline, liposomes, oil in water emulsions and others. A composition may further include a detergent to make the compound more bioavailable, e.g., octylglucoside, among others. The present invention is not limited by the selection of the carrier or detergent.

Pharmaceutical compositions of this invention may contain other active agents, such as conventional antibiotics, such as, for example, vancomycin [see, e.g., International Patent Application NO. WO98/40401, published Mar. 10, 1998, incorporated by reference herein], or a compound that potentiates an effect of another compound in a composition or combination. Alternatively, pharmaceutical compositions may be administered with other anti-pathogenic molecules or antibiotic compounds. Other compositions may contain a compound of the invention combined with another antibiotic(s), preferably with an antibiotic that acts on another target, such as, for example, an agent that interfere with cell wall formation, a protein synthesis inhibitor, or an antibiotic that affects cell membranes. For example, a compound of the invention may be combined with a beta-lactam, a cephalosporin, or a macrolide, among other antibiotics. Pharmaceutical compositions comprising a compound of the invention and a steroid may also be used in a method of treatment of the invention.

A pharmaceutical compositions may also be formulated to suit a selected route of administration, and may contain ingredients specific to a route of administration [see, e.g., Remington: The Science and Practice of Pharmacy, Vol. 2, 19^{th} edition (1995)]. The preparation of these pharmaceutically acceptable compositions, from a component provided herin, or another desired component, having appropriate pH isotonicity, stability and other conventional characteristics is within the skill of the art.

Compounds of this invention may be crystallized or recrystallized from solvents such as organic solvents. In such cases solvates may be formed. This invention includes within its scope stoichiometric solvates including hydrates as well as compounds containing variable amounts of water that may be produced by processes such as lyophilization, or in other ways.

Since the compounds of the invention are intended for use in pharmaceutical compositions it will readily be understood that they are each provided in substantially pure form, for example at least 60% pure, more suitably at least 75% pure and preferably at least 85%, especially at least 98% pure (% are on a weight for weight basis). Impure preparations of a compound of the invention may be used for preparing the more pure forms used in the pharmaceutical compositions; these less pure preparations of the compounds should contain at least 1%, more suitably at least 5% and preferably from 10 to 59% of a compound of the formula set forth herein or salt thereof.

Pharmaceutically acceptable derivatives of the a compound of the invention, for example, a free base form or their acid addition or quaternary ammonium salts, for example their salts with mineral acids, e.g. hydrochloric, hydrobromic or sulphuric acids, or organic acids, e.g. acetic, fumaric or tartaric acids, among others. Compounds of formula set forth herein may also be prepared as the N-oxide.

Certain of the compounds of a formula may exist in the form of optical isomers, e.g. diastereoisomers and mixtures of isomers in all ratios, e.g. racemic mixtures.

Still another embodiment of a composition of this invention is a disinfectant composition that can be employed to inhibit or kill pathogens or, when they are present on a non-living or living surface. For example, one such surface could be a biological sample, such as a tissue culture, a skin surface, culture of mammalian or other cells. Alternatively, the surface to be disinfected may be non-living, such as laboratory equipment, lab bench surfaces, or other household surfaces which typically are breeding grounds for certain pathogens. These disinfectant compositions may contain one or more of the compounds of this invention, along with other ingredients typical for the intended use. One of skill in the art may readily select additional ingredients suitable for compositions directed toward applications to living tissue, e.g., buffers, vs. those additional ingredients useful in solutions for non-living surfaces, e.g., detergents, surfactants, or other materials.

Such compositions may be designed for a variety of application methods including spraying the surface, coating the surface, soaking the surface, being implanted beneath the surface, and other things.

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

Glossary

The following definitions are provided to facilitate understanding of certain terms used frequently herein.

“Bodily material(s) means any material derived from an individual or from an organism infecting, infesting or inhabiting an individual, including but not limited to, cells, tissues and waste, such as, bone, blood, serum, cerebrospinal fluid, semen, saliva, muscle, cartilage, organ tissue, skin, urine, stool or autopsy materials.

“Disease(s)” means any disease caused by or related to infection by a bacteria, including, for example, otitis media, conjunctivitis, pneumonia, bacteremia, meningitis, sinusitis, pleural empyema and endocarditis, and epiglottitis and most particularly meningitis, such as for example infection of cerebrospinal fluid.

“Host cell(s)” is a cell that has been introduced (e.g., transformed or transfected) or is capable of introduction (e.g., transformation or transfection) by an exogenous polynucleotide sequence.

“Identity,” as known in the art, is a relationship between two or more polypeptide sequences or two or more polynucleotide sequences, as the case may be, as determined by comparing the sequences. In the art, “identity” also means the degree of sequence relatedness between polypeptide or polynucleotide sequences, as the case may be, as determined by the match between strings of such sequences. “Identity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well known Smith Waterman algorithm may also be used to determine identity.

Parameters for polypeptide sequence comparison include the following: Algorithm: Needleman and Wunsch, J. Mol. Biol. 48: 443-453 (1970)
Comparison matrix: BLOSSUM62 from Hentikoff and Hentikoff, Proc. Natl. Acad. Sci. USA. 89:10915-10919 (1992)
Gap Penalty: 12
Gap Length Penalty: 4

A program useful with these parameters is publicly available as the “gap” program from Genetics Computer Group, Madison Wis. The aforementioned parameters are the default parameters for peptide comparisons (along with no penalty for end gaps).

Parameters for polynucleotide comparison include the following: Algorithm: Needleman and Wunsch, J. Mol. Biol. 48: 443-453 (1970)
Comparison matrix: matches=+10, mismatch=0
Gap Penalty: 50
Gap Length Penalty: 3

Available as: The “gap” program from Genetics Computer Group, Madison Wis. These are the default parameters for nucleic acid comparisons.

A preferred meaning for “identity” for polynucleotides and polypeptides, as the case may be, are provided in (1) and (2) below.

(1) Polynucleotide embodiments further include an isolated polynucleotide comprising a polynucleotide sequence having at least a 95, 97 or 100% identity to the reference sequence of SEQ ID NO:1, wherein said polynucleotide sequence may be identical to the reference sequence of SEQ ID NO:1 or may include up to a certain integer number of nucleotide alterations as compared to the reference sequence, wherein said alterations are selected from the group consisting of at least one nucleotide deletion, substitution, including transition and transversion, or insertion, and wherein said alterations may occur at the 5′ or 3′ terminal positions of the reference nucleotide sequence or anywhere between those terminal positions, interspersed either individually among the nucleotides in the reference sequence or in one or more contiguous groups within the reference sequence, and wherein said number of nucleotide alterations is determined by multiplying the total number of nucleotides in SEQ ID NO:1 by the integer defining the percent identity divided by 100 and then subtracting that product from said total number of nucleotides in SEQ ID NO:1, or:
nₙ≦xₙ−(xₙ·y),
wherein nₙ is the number of nucleotide alterations, xₙ is the total number of nucleotides in SEQ ID NO:1, y is 0.95 for 95%, 0.97 for 97% or 1.00 for 100%, and · is the symbol for the multiplication operator, and wherein any non-integer product of xₙ and y is rounded down to the nearest integer prior to subtracting it from xₙ. Alterations of a polynucleotide sequence encoding the polypeptide of SEQ ID NO:2 may create nonsense, missense or frameshift mutations in this coding sequence and thereby alter the polypeptide encoded by the polynucleotide following such alterations.

(2) Polypeptide embodiments further include an isolated polypeptide comprising a polypeptide having at least a 95, 97 or 100% identity to a polypeptide reference sequence of SEQ ID NO:2, wherein said polypeptide sequence may be identical to the reference sequence of SEQ ID NO:2 or may include up to a certain integer number of amino acid alterations as compared to the reference sequence, wherein said alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence, and wherein said number of amino acid alterations is determined by multiplying the total number of amino acids in SEQ ID NO:2 by the integer defining the percent identity divided by 100 and then subtracting that product from said total number of amino acids in SEQ ID NO:2, or:
nₐ≦xₐ−(xₐ·y),
wherein na is the number of amino acid alterations, xₐ is the total number of amino acids in SEQ ID NO:2, y is 0.95 for 95%, 0.97 for 97% or 1.00 for 100%, and · is the symbol for the multiplication operator, and wherein any non-integer product of xₐ and y is rounded down to the nearest integer prior to subtracting it from xₐ.

“Individual(s)” means a multicellular eukaryote, including, but not limited to a metazoan, a mammal, an ovid, a bovid, a simian, a primate, and a human.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, as the term is employed herein. Moreover, a polynucleotide or polypeptide that is introduced into an organism by transformation, genetic manipulation or by any other recombinant method is “isolated” even if it is still present in said organism, which organism may be living or non-living.

“Pathogen(s/ic)” means a prokaryotic pathogen or eukaryotic pathogen, none or which is necessarily a pathogen in the medical or scientific sense known in the art, and (A) wherein “prokaryotic pathogen(s)” means (i) any bacteria capable of infecting, infesting or living on or in a vertebrate, including, but not limited to a member of the genus Streptococcus, Staphylococcus, Bordetella, Corynebacterium, Mycobacterium, Neisseria, Haemophilus, Actinomycetes, Streptomycetes, Nocardia, Enterobacter, Yersinia, Fancisella, Pasturella, Moraxella, Acinetobacter, Erysipelothrix, Branhamella, Actinobacillus, Streptobacillus, Listeria, Calymmatobacterium, Brucella, Bacillus, Clostridium, Treponema, Escherichia, Salmonella, Kleibsiella, Vibrio, Proteus, Erwinia, Borrelia, Leptospira, Spirillum, Campylobacter, Shigella, Legionella, Pseudomonas, Aeromonas, Rickettsia, Chlamydia, Borrelia and Mycoplasma, and further including, but not limited to, a member of the species or group, Group A Streptococcus, Group B Streptococcus, Group C Streptococcus, Group D Streptococcus, Group G Streptococcus, Streptococcus pneumoniae, Streptococcus pyogenes, Streptococcus agalactiae, Streptococcus faecalis, Streptococcus faecium, Streptococcus durans, Neisseria gonorrheae, Neisseria meningitidis, Staphylococcus aureus, Staphylococcus epidermidis, Corynebacterium diptheriae, Gardnerella vaginalis, Mycobacterium tuberculosis, Mycobacterium bovis, Mycobacterium ulcerans, Mycobacterium leprae, Actinomyctes israelii, Listeria monocytogenes, Bordetella pertusis, Bordatella parapertusis, Bordetella bronchiseptica, Escherichia coli, Shigella dysenteriae, Haemophilus influenzae, Haemophilus aegyptius, Haemophilus parainfluenzae, Haemophilus ducreyi, Bordetella, Salmonella typhi, Citrobacter freundii, Proteus mirabilis, Proteus vulgaris, Yersinia pestis, Kleibsiella pneumoniae, Serratia marcessens, Serratia liquefaciens, Vibrio cholera, Shigella dysenterii, Shigella flexneri, Pseudomonas aeruginosa, Franscisella tularensis, Brucella abortis, Bacillus anthracis, Bacillus cereus, Clostridium perfringens, Clostridium tetani, Clostridium botulinum, Treponema pallidum, Rickettsia rickettsii, Helicobacter pylori and Chlamydia trachomitis, (ii) an archaeon, including but not limited to Archaebacter, and (iii) a unicellular or filamentous eukaryote, including but not limited to, a protozoan, a fungus, a member of the genus Saccharomyces, Kluveromyces, or Candida, and a member of the species Saccharomyces ceriviseae, Kluveromyces lactis, or Candida albicans, and further including, but not limited to, gram negative bacteria. Specifically, however, the compounds of the present invention exert inhibitory activity on the biological activity of DXR reductoisomerase of the following clinically relevant bacteria: E. coli, and Haemophilus influenzae. However, it is to be understood by one of skill in the art that DXR reductoisomerase of bacterial species other than those listed herin are encompassed by this invention.

“Polynucleotide(s)” generally refers to any polyribonucleotide or polydeoxyribonucleotide, that may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that comprise one or more modified bases. Thus, DNAs or RNAs with backbones modified for stability or for other reasons are “polynucleotide(s)” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. “Polynucleotide(s)” also embraces short polynucleotides often referred to as oligonucleotide(s).

“Polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may comprise amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to those of skill in the art. It will be appreciated that the same type of modification may be present in the same or varying degree at several sites in a given polypeptide. Also, a given polypeptide may comprise many types of modifications. Modifications can occur anywhere in a polypeptide, including the peptide backbone, the amino acid side-chains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, GPI anchor formation, hydroxylation, iodination, methylation, myristoylation, oxidation, proteolytic processing, phosphorylation, prenylation, racemization, glycosylation, lipid attachment, sulfation, gamma-carboxylation of glutamic acid residues, hydroxylation and ADP-ribosylation, selenoylation, sulfation, transfer-RNA mediated addition of amino acids to proteins, such as arginylation, and ubiquitination. See, for instance, PROTEINS—STRUCTURE AND MOLECULAR PROPERTIES, 2nd Ed., T. E. Creighton, W. H. Freeman and Company, New York (1993) and Wold, F., Posttranslational Protein Modifications: Perspectives and Prospects, pgs. 1-12 in POSTTRANSLATIONAL COVALENT MODIFICATION OF PROTEINS, B. C. Johnson, Ed., Academic Press, New York (1983); Seifter et al., Meth. Enzymol. 182:626-646 (1990) and Rattan et al., Protein Synthesis: Posttranslational Modifications and Aging, Ann. N.Y. Acad. Sci. 663: 48-62 (1992). Polypeptides may be branched or cyclic, with or without branching. Cyclic, branched and branched circular polypeptides may result from post-translational natural processes and may be made by entirely synthetic methods, as well.

“Recombinant expression system(s)” refers to expression systems or portions thereof or polynucleotides of the invention introduced or transformed into a host cell or host cell lysate for the production of the polynucleotides and polypeptides of the invention.

“Variant(s)” as the term is used herein, is a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusion proteins and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. The present invention also includes include variants of each of the polypeptides of the invention, that is polypeptides that vary from the referents by conservative amino acid substitutions, whereby a residue is substituted by another with like characteristics. Typical such substitutions are among Ala, Val, Leu and Ile; among Ser and Thr; among the acidic residues Asp and Glu; among Asn and Gln; and among the basic residues Lys and Arg; or aromatic residues Phe and Tyr. Particularly preferred are variants in which several, 5-10, 1-5, 1-3, 1-2 or 1 amino acids are substituted, deleted, or added in any combination. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to skilled artisans.

“Inhibition,” “inhibiting,” to “inhibitory” means with reference to a mechanism of action, enzyme activity, enzyme, or polynucleotide herein (i) altering, modulating, lowering, diminishing, preventing or stopping an activity of an enzyme, including, but not limited to a reductoisomerase or a DXR enzyme, or (ii) altering, modulating, lowering, diminishing, preventing or stopping an activity of an enzyme, including, but not limited to the biosynthesis of either menaquinone or ubiquinone or any other compound synthesized by a DXR enzyme, or (iii) altering, modulating, lowering, diminishing, preventing or stopping DXR dimer formation.

“Activity” means any activity of DXR of the invention, including but not limited to a biological activity of DXR, an enzymatic activity of DXR, a protein-protein interaction comprising DXR, formation of a dimer, use of manganese as a substrate by DXR, use of NADPH as substrate by DXR, NADPH binding to DXR prior to or simultaneous with manganese binding, isomerization of substrate, binding of substrate, reductoisomerizations, binding of DXR with a cellular component, conversion of 1-deoxy-D-xylulose-5-phosphate to 2C-methyl-D-erythritol-4-phosphate, conversion of NADPH to NADP, inhibition of DXR by fosmidomycin, fosfomycin, FR-33289, or FR-900098, or binding of fosmidomycin, fosfomycin, FR-33289, or FR-900098 to DXR.

### EXAMPLES

The examples below are carried out using standard techniques, that are well known and routine to those of skill in the art, except where otherwise described in detail. The examples are illustrative, but do not limit the invention.

### Example 1

### Strain Selection, Library Production and Sequencing

The polynucleotide having a DNA sequence given in Table 1 [SEQ ID NO:1] was obtained from a library of clones of chromosomal DNA of H. influenzae. The sequencing data from two or more clones comprising overlapping H. influenzae DNAs was used to construct the contiguous DNA sequence in SEQ ID NO:1. Libraries may be prepared by routine methods, for example:

Methods 1 and 2 below.

Total cellular DNA is isolated from H. influenzae according to standard procedures and size-fractionated by either of two methods.

Method 1

Total cellular DNA is mechanically sheared by passage through a needle in order to size-fractionate according to standard procedures. DNA fragments of up to 11 kbp in size are rendered blunt by treatment with exonuclease and DNA polymerase, and EcoRI linkers added. Fragments are ligated into the vector Lambda ZapII that has been cut with EcoRI, the library packaged by standard procedures and E. coli infected with the packaged library. The library is amplified by standard procedures.

Method 2

Total cellular DNA is partially hydrolyzed with a one or a combination of restriction enzymes appropriate to generate a series of fragments for cloning into library vectors (e.g., RsaI, PalI, AluI, Bshl2351), and such fragments are size-fractionated according to standard procedures. EcoRI linkers are ligated to the DNA and the fragments then ligated into the vector Lambda ZapII that have been cut with EcoRI, the library packaged by standard procedures, and E. coli infected with the packaged library. The library is amplified by standard procedures.

### Example 2

### Mechanism of Action of E. coli DXR

DXR is a target of the antibiotic fosmidomycin. This was demonstrated by overexpression of E. coli DXR in E. coli cells (DH5α). The MIC of fosmidomycin in this experiment was increased 8-fold for cells overexpressing E. coli DXR. These data demonstrate that DXR is an antibacterial target for this antibiotic in E. coli.

The E. coli dxr gene was cloned via techniques well known to those skilled in the art. The E. coli dxr gene clone was used in the minimum inhibitory concentration (herein “MIC”) experiment below in Table 2. E. coli dxr was amplified by PCR using E. coli chromosome DNA as template. The primers for E. coli dxr are 5′-TTATACATATGAAGCAACTCACCATTCTGGGC-3′ [SEQ ID NO:3] and 5′-TTATAGGATCCTCAGCTTGCGAGACGCATCACCTC-3′ [SEQ ID NO:4]. The PCR conditions are: 100 μl volume, 400 ng template DNA, 0.2 mM dNTP, 0.25 pM primers, 10 μl 10× particle forming units (herein “pfu”) reaction buffer and 2.5 units of pfu DNA polymerase. The PCR cycle conditions were: 25 cycles at 94° C. for 60 seconds, 55° C. for 40 seconds, and 72° C. for 80 seconds. The PCR DNA was purified using Qiagen's PCR purification kit. The plasmid pET28a(+) and E. coli dxr PCR fragments were both digested using both NdeI and BamHI. The ligation of the plasmid and PCR fragments were at 15° C. overnight. The ligation reaction was then transformed into E. coli DH5α and colonies were selected using LB plates containing ampicillin. The selected colonies were grown to a sufficient optical density to extract plasmid DNA using minipreps. In addition, the isolated plasmid was sequenced to confirm the insertion of the E. coli dxr gene in the plasmid.

MIC experiments revealed that the mode of action of fosmidomycin is DXR in E. coli. The E. coli DXR MIC in Table 2 below shows the results. The MIC protocol used techniques well known to those skilled in the art. All strains were grown overnight in LB with appropriate selection. The compounds are mixed or put in contact with DMSO. Fifty μl of LB was placed in each well of the microtiter plate, except in Column 1. To obtain a top concentration of 256 μl/ml of compound after the inoculum is added: 10 μl of a 5.12 mg/ml stock is mixed with 90 μl of LB. This mixture was placed in the first well of its assigned row of a 96-well plate. The compound was then serially diluted 1:2 by taking 50 μl from the first well in the row and mixing it with the second well of the same row, Column 2. Then, 50 μl from that well was mixed with the third well in the row, Column 3 and so on until Column 11. Fifty μl of the serial dilution from Column 11 was discarded. The final well in the row, column 12, was the growth control. No compound was placed in column 12. The overnight culture was diluted 1:50 into LB. Fifty μl of this culture dilution was added to every well of the microtiter plate. This dilutes the inoculum and drug 1:2. The final inoculum was approximately 1×10⁵ cells/ml. The 96-well pates are then incubated overnight at 37° C. The following day the MICs for each compound were recorded as the first well in the row that did not contain any visible bacterial growth.

TABLE 2
E. coli DXR MICs (μg/ml)

| | E. coli | E. coli DH5α + | E. coli DH5α + |
|---|---|---|---|
| | DH5α | (pET28) | (pET28::dxr) |
| Fosmidomycin | 16 | 16 | 128 |
| Amoxicillin | 16 | 16 | 16 |
| Ciprofloxacin | 0.006 | 0.006 | 0.006 |
| Tetracycline | 16 | 16 | 16 |

Mechanism of Action of H. influenzae DXR

DXR as the target of the antibiotic fosmidomycin in H. influenzae was demonstrated by the inventors through overexpression of H. influenzae DXR in E. coli cells (DH5α). The MIC of fosmidomycin in this experiment was increased 16-fold for cells expressing H. influenzae DXR. These data demonstrate that DXR is an antibacterial target for this antibiotic in H. influenzae.

The H. influenzae dxr gene was cloned via techniques well known to those skilled in the art. The H. influenzae dxr gene clone was used in the minimum inhibitory concentration experiment below in Table 3. H. influenzae dxr was amplified by PCR using E. coli chromosome DNA as template. The primers for H. influenzae dxr are 5′-TATATACATATGCAAAAACAAAACATTGTCATTC-3′ [SEQ ID NO:5] and 5′-TATATAGGATCCTCATTCTCTAAGTAGTGTTTTCGCAATC-3′ [SEQ ID NO:6]. The PCR conditions were: 100 μl volume, 400 ng template DNA, 0.2 mM dNTP, 0.25 pM primers, 10 μl 10× pfu reaction buffer and 2.5 units of pfu DNA polymerase. The PCR cycle conditions were: 25 cycles at 94° C. for 60 seconds, 55° C. for 40 seconds, and 72° C. for 80 seconds. The PCR DNA was purified using Qiagen's PCR purification kit. The plasmid pET28a(+) and H. influenzae dxr PCR fragments were both digested using both NdeI and BamHI. The ligation of the plasmid and PCR fragments were at 15° C. overnight. The ligation reaction was then transformed into E. coli DH5α and colonies were selected using LB plates containing ampicillin. The selected colonies were grown to a sufficient optical density to extract plasmid DNA using minipreps. In addition, the isolated plasmid was sequenced to confirm the insertion of the H. influenzae dxr gene in the plasmid.

MIC experiments suprisingly revealed that the mode of action of fosmidomycin is DXR in H. influenzae. H. influenzae DXR MIC in Table 3 below shows the results. The MIC protocol used techniques well known to those skilled in the art. All strains were grown overnight in LB with appropriate selection. The compounds are mixed or put in contact with DMSO. Fifty μl of the LB was placed in each well of the microtiter plate, except in Column 1. To obtain a top concentration of 256 μg/ml of compound after the inoculum is added: 10 μl of a 5.12 mg/ml stock is mixed with 90 μl of LB. This mixture was placed in the first well of its assigned row of a 96-well plate. The compound was then serially diluted 1:2 by taking 50 μl from the first well in the row and mixing it with the second well of the same row, Column 2. Then, 50 μl from that well was mixed with the third well in the row, Column 3 and so on until Column 11. Fifty μl of the serial dilution from Column 11 was discarded. The final well in the row, column 12, was the growth control. No compound was placed in column 12. The overnight H. influenzae culture was diluted 1:50 into LB. Fifty μl of this culture dilution was added to every well of the microtiter plate. This dilutes the inoculum and drug 1:2. The final inoculum was approximately 1×10⁵ cells/ml. The 96-well pates are then incubated overnight at 37° C. The following day the MICs for each compound were recorded as the first well in the row that did not contain any visible bacterial growth.

TABLE 3
H. influenzae DXR MICs (μg/ml)

| | | E. coli DH5α + | E. coli DH5α + |
|---|---|---|---|
| | E. coli DH5α | (pET28) | (pET28::dxr) |
| Fosmidomycin | 16 | 16 | 256 |
| Amoxicillin | 16 | 16 | 16 |
| Ciprofloxacin | 0.006 | 0.006 | 0.006 |
| Tetracycline | 16 | 16 | 16 |

1. A method of modulating an activity of a DXR reductoisomerase enzyme of Haemophilus influenzae comprising contacting said enzyme with a compound that modulates an activity of said enzyme.

2. The method according to claim 1, wherein said activity is selected from the group consisting of:
formation of a dimer;
use of manganese as a substrate by DXR;
use of NADPH as substrate by DXR;
NADPH binding to DXR prior to or simultaneous with manganese binding;
isomerization of substrate;
binding of substrate;
reductoisomerizations;
binding of DXR with a cellular component;
conversion of 1-deoxy-D-xylulose-5-phosphate to 2C-methyl-D-erythritol-4-phosphate;
conversion of NADPH to NADP;
inhibition of DXR by fosmidomycin, fosfomycin, FR-33289, or FR-900098; and
binding to DXR of fosmidomycin, fosfomycin, FR-33289, or FR-900098 to DXR;

3. The method according to claim 1, wherein said modulating is inhibiting.

4. The method according to claim 1, wherein contacting said enzyme with said compound inhibits the biosynthesis of isoprenoids.

5. The method according to claim 1, wherein contacting said enzyme with said compound inhibits the biosynthesis of either menaquinone or ubiquinone or biosynthesis of both menaquinone and ubiquinone.

6. The method of claim 1, wherein said Haemophilus influenzae DXR reductoisomerase enzyme is selected from the group consisting of:
(i) an polypeptide comprising the amino acid sequence of SEQ ID NO:2,
(ii) an polypeptide that is the amino acid sequence of SEQ ID NO:2, and
(iii) a polypeptide encoded by a polynucleotide comprising the polynucleotide sequence of SEQ ID NO:1.

7. The method according to claim 1, wherein said compound forms a stable complex comprising said enzyme and said compound.

8. The method according to claim 1, wherein contacting with said compound kills or inhibits replication of a Haemophilus influenzae carrying said enzyme.

9. The method according to claim 8, wherein said contacting step occurs in vitro.

10. The method according to claim 8, wherein said contacting step occurs in a mammal infected with said Haemophilus influenzae.

11. The method according to claim 8, wherein said contacting step occurs ex vivo.

12. A method for treating a mammal or mammalian tissue infected with Haemophilus influenzae comprising a DXR reductoisomerase enzyme, said method comprising administering to said mammal an effective amount of a pharmaceutical composition comprising a compound that inhibits a Haemophilus influenzae DXR reductoisomerase enzyme in a pharmaceutically or physiologically acceptable carrier.

13. The method according to claim 12 wherein said composition is administered by a route selected from intravenous, oral, intradermal, transdermal, intraperitoneal, intramuscular, subcutaneous, inhalation, and mucosal.

14. The method according to claim 12, wherein an effective amount of said compound comprises about 1 mg to 500 mg.

15. The method according to claim 12, wherein said mammal is a human.

16. The method according to claim 12, wherein said mammal is a domestic animal.

17. A method for disinfecting a surface comprising contacting said surface with a composition comprising a compound which inhibits a Haemophilus influenzae DXR reductoisomerase enzyme.

18. The method according to claim 17, wherein said surface is a biological tissue.

19. The method according to claim 17, wherein said surface is part of a non-living structure.

20. The method according to claim 17, wherein said contacting step comprises administering a suitable disinfecting dosage of said composition by means selected from the group consisting of coating, spraying, implanting, and soaking.
